(19) United States
(12) Patent Application Publication
(10) Pub. No.: US 2010/0137976 A1
(43) Pub. Date: Jun. 3, 2010

(54) Systems and Methods for Treating Heart Tissue Via Localized Delivery of Parp Inhibitors

(75) Inventors: Carol Sullivan, Petaluma, CA (US); Ayala Hezi-Yamit, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/326,609

(22) Filed: Dec. 2, 2008

(51) Int. Cl. A61K 31/4985; A61K 31/52; A61F 2/82; A61K 31/7088; A61K 38/18; A61K 39/12; A61K 31/728; A61K 39/395; A61K 38/43; A61P 9/00

(52) U.S. Cl. 623/1.42; 514/252.16; 514/263.1; 514/12; 424/93.6; 514/53; 424/130.1; 424/94.1; 514/44 A; 514/44 R

(57) ABSTRACT

The systems and methods of the present disclosure, in a broad aspect, provide for treatment of cardiac tissue via localized delivery of PARP inhibitors. These systems include a composition comprising at least one poly(ADP-ribose) polymerase (PARP) inhibitor; and at least one delivery device for introducing the composition into the cardiac tissue.

### FIELD OF THE INVENTION

The present disclosure generally relates to systems and associated methods for delivering at least one poly(ADP-ribose) polymerase (PARP) inhibitor to cardiac tissue for the treatment of diseases and conditions.

### BACKGROUND OF THE INVENTION

The human heart wall consists of an inner layer of simple squamous epithelium, referred to as the endocardium, overlying a variably thick heart muscle or myocardium and is enveloped within a multi-layer tissue structure referred to as the pericardium. The innermost layer of the pericardium, referred to as the visceral pericardium or epicardium, covers the myocardium. The epicardium reflects outward at the origin of the aortic arch to form an outer tissue layer, referred to as the parietal pericardium, which is spaced from and forms an enclosed sac extending around the visceral pericardium of the ventricles and atria. An outermost layer of the pericardium, referred to as the fibrous pericardium, attaches the parietal pericardium to the sternum, the great vessels and the diaphragm so that the heart is confined within the middle mediastinum. Normally, the visceral pericardium and parietal pericardium lie in close contact with each other and are separated only by a thin layer of a serous pericardial fluid that enables friction free movement of the heart within the sac. The space between the visceral and parietal pericardia is referred to as the pericardial space. In common parlance, the visceral pericardium is usually referred to as the epicardium, and epicardium will be used hereafter. Similarly, the parietal pericardium is usually referred to as the pericardium, and pericardium will be used hereafter in reference to parietal pericardium.

Heart disease, including myocardial infarction (MI), is a leading cause of death and disability in human beings, particularly in the western world, most particularly among males. A variety of heart diseases can progress to heart failure by a common mechanism called remodeling. With remodeling, cardiac function progressively deteriorates, often leading to clinical heart failure and associated symptoms. Heart disease can in turn impair other physiological systems. Each year over 1.1 million Americans have a myocardial infarction (MI). Myocardial infarction can result in an acute depression in ventricular function and expansion of the infarcted tissue under stress. This triggers a cascading sequence of myocellular events known as remodeling. In many cases, this progressive myocardial infarct expansion and remodeling leads to deterioration in ventricular function and heart failure. Such ischemic cardiomyopathy is the leading cause of heart failure in the United States. It is the objective of the present invention to improve vascular supply to patients who have or are at high-risk of developing cardiac disease (such as cardiac ischemia). Acutely or chronically diseased cardiac tissue would benefit from increased blood supply. Studies have shown that even in the adult, normal repair mechanisms are elicited (e.g. those involving the recruitment of endogenous regenerative cells) following cardiac injury. Inadequate blood supply limits the survival of such cells and may prevent healing. Blood supply is required to bring necessary oxygen, nutrients, and blood components (cells, chemokines, etc.) to the injured region and to clear metabolic products. A treatment that improves blood supply to such a region is very likely to benefit the patient by facilitating greater recovery.

Cardiac tissue can be acutely or chronically ischemic. Severe ischemia resulting in cardiac cell death is referred to as infarction. Acute or chronic recovery may be improved by increasing vascular supply to or around the affected injured region.

A stenosed or blocked coronary artery is one example of heart disease. A completely or substantially blocked coronary artery can cause immediate, intermediate term, and/or long-term adverse effects. In the immediate term, a myocardial infarction can occur when a coronary artery becomes occluded and can no longer supply blood to the myocardial tissue, thereby resulting in myocardial cell death. When a myocardial infarction occurs, the myocardial tissue that is no longer receiving adequate blood flow dies and is eventually replaced by scar tissue.

Within seconds of a myocardial infarction, the under-perfused myocardial cells no longer contract, leading to abnormal wall motion, high wall stresses within and surrounding the infarct, and depressed ventricular function. The high stresses at the junction between the infarcted tissue and the normal tissue lead to expansion of the infarcted area and to remodeling of the heart over time. These high stresses injure the still viable myocardial cells and eventually depress their function. This results in an expansion of injury and dysfunctional tissue including and beyond the original myocardial infarct region.

According to the American Heart Association, in the year 2000 approximately 1,100,000 new myocardial infarctions occurred in the United States. For 650,000 patients this was their first myocardial infarction, while for the other 450,000 patients this was a recurrent event. Two hundred-twenty thousand people suffering MI die before reaching the hospital. Within one year of the myocardial infarction, 25% of men and 38% of women die. Within 6 years, 22% of men and 46% of women develop heart failure, of which 67% are disabled. This is despite modern medical therapy.

The consequences of myocardial infarction are often severe and disabling. When a myocardial infarction occurs, the myocardial tissue that is no longer receiving adequate blood flow dies and is replaced with scar tissue. This infarcted tissue cannot contract during systole, and may actually undergo lengthening in systole and leads to an immediate depression in ventricular function. This abnormal motion of the infarcted tissue can cause delayed or abnormal conduction of electrical activity to the still surviving peri-infarct tissue (tissue at the junction between the normal tissue and the infarcted tissue) and also places extra structural stress on the peri-infarct tissue.

The zone receiving the reduced blood flow is known as an ischemic zone. Furthermore, the elevation of matrix metalloproteinases, reduction in tissue inhibitors of the matrix metalloproteinases (TIMPs), and consequent degradation of collagen may play an additional role in ischemic cardiomyopathy. To improve cardiac function in patients with ischemic cardiomyopathies, there is a need to re-establish blood flow to the ischemic zones.

In addition to immediate hemodynamic effects, the infarcted heart tissue undergoes three major processes: infarct expansion, infarct extension, and chamber remodeling. These factors individually and in combination contribute to the eventual dysfunction observed in the cardiac tissue remote from the site of the infarction

Infarct expansion is a fixed, permanent, disproportionate regional thinning and dilatation of tissue within the infarct zone. Infarct expansion occurs early after a myocardial infarction. The mechanism is slippage of the tissue layers.

Infarct extension is additional myocardial necrosis following myocardial infarction. Infarct extension results in an increase in total mass of infarcted tissue and the additional infarcted tissue may also undergo infarct expansion. Infarct extension occurs days after a myocardial infarction. The mechanism for infarct extension appears to be an imbalance in the blood supply to the peri-infarct tissue versus the increased oxygen demands on the tissue.

The mechanisms which lead to cardiac tissue injury and organ dysfunction especially after ischemia and/or reperfusion are multiple. Less targeted, systemic delivery may send a therapeutic composition or agent to an unintended part of the body and actually cause harm. This is possible because of the body's circulatory system There is an unmet need in the art to cardiac tissue to prevent injury, during injury or after an injurious event has occurred which avoids the disadvantages of systemic delivery of therapeutic compositions and agents.

### SUMMARY OF THE INVENTION

These and other objects are achieved by the systems and methods of the present disclosure, which in a broad aspect, treat cardiac tissue before, after or during injury.

In one embodiment, the present disclosure relates to a system for treating cardiac tissue comprising a composition comprising at least one poly(ADP)-ribose) polymerase (PARP) inhibitor and at least one delivery device for introducing the composition into the cardiac tissue.

In another embodiment of the present system, the at least one PARP inhibitor is a PARP-1 inhibitor or a PARP-2 inhibitor. Alternatively, the at least one PARP inhibitor may be INO-1001 or BGP-15.

In another embodiment of the present system, the cardiac tissue is previously injured.

In another embodiment of the present system, the composition is introduced to the cardiac tissue at the location of and during revascularization

In another embodiment of the present system, the composition is introduced into the cardiac tissue during an injurious event or after an injurious event has occurred.

In another embodiment of the present system, the cardiac tissue is selected from the group consisting of injured cardiac tissue, peri-injured cardiac tissue, and healthy cardiac tissue surrounding injured cardiac tissue.

In another embodiment of the present system, the delivery device is an injection catheter selected from the group consisting of an endocardial injection catheter, a transvacular injection catheter, and an epicardial injection catheter. In another embodiment of the present system, the delivery device is a stent or stent graft.

In another embodiment of the present system, the composition further comprises a bioactive agent.

In another embodiment of the present system, the introduction of the composition into the injured cadiac tissue is via an injection site on the injured cardiac tissue.

In another embodiment of the present system, the injection site in the injured cardiac tissue is selected from the group consisting of sub-endocardial, sub-epicardial and intra-myocardial sites.

In another embodiment of the present system, the bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof.

In another embodiment of the present system, the composition further comprises a contrast agent.

In another embodiment of the present system, the composition is provided to the injured cardiac tissue between about 1 hour and about 1 year after injury occurs to the cardiac tissue.

In another embodiment of the present system, the composition is provided in about 1 to 20 injections.

In another embodiment of the present system, injections are provided sequentially.

In another embodiment of the present system, injections are provided approximately simultaneously.

The present disclosure also relates to a method for treating cardiac tissue comprising delivering a composition comprising at least one poly(ADP-ribose) polymerase (PARP) inhibitor to the cardiac tissue of a patient in need thereof with at least one delivery device for introducing the composition into the cardiac tissue.

In another embodiment of the present method, the at least one PARP inhibitor is PARP-1 inhibitor or a PARP-2 inhibitor. Alternatively, the at least one PARP inhibitor may be INO-1001 or BGP-15.

In another embodiment of the present method, the cardiac tissue is previously injured.

In another embodiment of the present method, the composition is introduced into the cardiac tissue at the location of and during revascularization

In another embodiment of the present method, the composition is introduced into the cardiac tissue during an injurious event or after an injurious event has occurred.

In another embodiment of the present method, the cardiac tissue is selected from the group consisting of injured cardiac tissue, peri-injured cardiac tissue, and healthy cardiac tissue surrounding injured cardiac tissue.

In another embodiment of the present method, the delivery device is an injection catheter selected from the group consisting of an endocardial injection catheter, a transvacular injection catheter and an epicardial injection catheter. In another embodiment of the present method, the delivery device may be a stent or stent graft.

In another embodiment of the present method, the composition further comprises a bioactive agent.

In another embodiment of the present method, the introduction of the composition into the injured cadiac tissue is via an injection site on the injured cardiac tissue.

In another embodiment of the present method, the injection site in the injured cardiac tissue is selected from the group consisting of sub-endocardial, sub-epicardial and intra-myocardial sites.

In another embodiment of the present method, the bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof.

In another embodiment of the present method, the composition further comprises a contrast agent.

In another embodiment of the present method, the composition is provided to the injured cardiac tissue between about 1 hour and about 1 year after injury occurs to the cardiac tissue.

In another embodiment of the present method, the composition is provided in about 1 to 20 injections.

In another embodiment of the present method, injections are provided sequentially.

In another embodiment of the present method, injections are provided approximately simultaneously.

### DETAILED DESCRIPTION OF THE INVENTION

The present disclosures provides apparatus and associated methods for local delivery of at least one poly(ADP-ribose) polymerase (PARP) inhibitor with at least one delivery device for introducing the composition into the cardiac tissue.

The mechanisms leading to cardiac tissue injury and/or organ dysfunction especially after ischemia/reperfusion or hypoxia/reoxygenation are multiple. There is evidence that reactive oxygen species contribute to reperfusion injury in the previously ischemic myocardium which, in turn, leads to PARP activation with subsequent myocardial and vascular injury resulting.

Under physiological conditions, one of ordinary skill in the art knows that PARP is constitutively activated at low levels and plays a role in housekeeping DNA repair functions. Under pathological conditions however, the DNA damage may excessive, for example, from acute oxidant stress induced by physical trauma, toxin exposure, or ischemia-reperfusion. Under these conditions, PARP may be over-activated. PARP may enzymatically deplete its substrate, nicotinamide adenine dinucleotide (NAD) which is a critical molecule for cellular metabolism. NAD is an obligatory cofactor for substrate-level phosphorylation (glycolysis) and oxidative phosphorylation (Krebs cycle, electron transport chain).

In the absence of adequate levels of intracellular NAD, the cells cannot form ATP and therefore eventually will under necrosis. In contrast to apoptosis, cell death by necrosis leads to the expulsion of cellular contents. This leads to inflammation and further damage to surrounding tissue. PARP inhibition blocks oxidant-mediated NAD depletion and preserves cellular ATP, thereby, preventing necrosis. This may protect tissues and organs from oxidant-mediated infarction.

The at least one PARP inhibitor which is delivered to a cardiac tissue may be for example a PARP-1 or PARP-2 inhibitor in another embodiment. Moreover, it is within the scope and teaching of the present disclosure to cover additional various PARP inhibitors.

One such PARP inhibitor is BGP-15 by N-Gene Research Laboratories, Inc. BGP-15 is an insulin sensitizer and PARP inhibitor with a mechanism of action, which is designed to restore cNOS and inducible heat shock protein (HSP) functions resulting in correction of impaired functions of mitochondria. Another is INO-1001 by Inotek Pharmaceuticals Inc. It is a potent inhibitor of the nuclear enzyme poly(ADP ribose) polymerase (PARP), which can be considered a target for diseases mediated by cell necrosis, DNA repair abnormality, and inflammation. Under normal conditions, PARP is involved in the repair of DNA single strand breaks caused by oxidative stress via the activation and recruitment of DNA repair enzymes in the nucleus. Under conditions where DNA damage is excessive (such as by acute excessive exposure to a pathological insult), PARP is over-activated, resulting in cell-based energetic failure characterized by NAD depletion and leading to ATP consumption, cellular necrosis, tissue injury, and organ damage/failure.

The present PARP inhibitors may be delivered locally. The direct or selective delivery of agents to cardiac tissue is often preferred over systemic delivery of such agents for several reasons. One reason is the substantial expense and small amount of the medical agents available. Another reason is the substantially great concentration of such agents that can be delivered directly to cardiac tissue, compared with the dilute concentrations possible through systemic delivery. Another reason is that systemic administration is associated with systemic toxicity at doses required to achieve desired drug concentrations in the cardiac tissue.

In the absence of adequate blood flow in the injured region, endogeous repair mechanisms are not able to restore cardiac tissue or function. Endogenous cells have been demonstrated to home to injured tissue, even in the adult heart, but blood flow limitations may prevent them from taking residence and promote healing.

Progressive deterioration in heart function can occur initially in the absence of symptoms. Eventually, however, symptoms of clinical heart failure develop, such as shortness of breath, swelling, difficulty breathing in the supine position, arrhythmias, and even organ failure. Even patients with asymptomatic cardiac dysfunction and milder forms of heart failure are at increased risk of sudden cardiac death.

Before any composition is injected into a heart having a region of injured tissue, to treat the tissue with at least one PARP inhibitor, the location and extent of the injured region is identified. Multiple technologies and approaches are available for the clinician to identify and assess normal, injured non-viable, and injured-viable cardiac tissue. These include, but are not limited to, visual inspection during open chest surgical procedures, localized blood flow determinations, local electrical and structural activity, nuclear cardiology, echocardiography, echocardiographic stress test, coronary angiography, magnetic resonance imaging (MRI), computerized tomography (CT) scans, and ventriculography.

Once the location, size and shape of the injured region are identified, the clinician can access and begin delivery to the cardiac wall the at least one PARP inhibitors within the scope and teachings of the present disclosure. The cardiac tissue(s) to which the at least one PARP inhibitor may be cardiac tissue that was previously injured. This injury may have caused overactivation of PARP so that inhibition of it may bring treatment to the cardiac tissue(s). In another embodiment, PARP inhibitors may be delivered to cardiac tissue during an injurious event or after an injurious event has occurred. These are also times when the cardiac tissue(s) needs PARP inhibition to prevent cell necrosis.

Revascularization is a surgical procedure for the provision of a new, additional, or augmented blood supply to a body part or organ. The term derives from the prefix re-, in this case meaning “restoration” and vasculature, which refers to the circulatory structures of an organ. Revascularization involves a thorough analysis and diagnosis and treatment of the existing diseased vasculature of the affected organ, and can be aided by the use of different imaging modalities such as magnetic resonance imaging, PET scan, CT scan, and X ray fluoroscopy. This is a concept important in the subdisciplines of biomedicine which are concerned with the rehabilitation of important organs, such as the heart, liver, and lungs. Revascularization can performed following an ischemic event. In accordance with the systems and methods of the present disclosure, the present PARP inhibitors may be delivered to the location of and/or at the time of revacularization.

The PARP inhibitors in accordance with the scope and teachings of the present disclosure can be effective in treating or preventing acute myocardial infarction, serve as cardioprotectant prior to surgical interventions including angioplasty after acute myocardial infarction and cardiopulmonary bypass (CPB) surgery and aortic aneurysm repair surgery.

Also, the composition containing at least one PARP inhibitor may include one or more bioactive agents to induce healing or regeneration of damaged cardiac tissue. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. The composition containing at least one PARP inhibitor may also include cellular additives such as stem cells, leukocytes, red blood cells, cultured cardiac cells, or other differentiated or undifferentiated cells.

Furthermore, the compositions containing at least one PARP inhibitor according to the present disclosure may include a contrast agent for detection by X-rays, magnetic resonance imaging (MRI) or ultrasound. Suitable contrast agents are known to persons of ordinary skill in the art and include, but are not limited to, radiopaque agents, echogenic agents and paramagenetic agents. A contrast agent may be used in composition of some embodiments for visual confirmation of injection success. Examples of such contrast agents include, but are not limited, X-ray contrast (e.g. IsoVue or other contrast agents having a high X-ray attenuation coefficient), MRI contrast (e.g., gadolinium or other contrast agents detectable as signal or signal-void by MRI), and ultrasound contrast (echogenic or echo-opaque compounds).

In order to practice the present invention and deliver a composition containing at least one PARP inhibitor to target sites within the cardiac wall, a clinician may use one of a variety of access techniques. These include surgical (sternotomy, thoracotomy, mini-thoracotomy, sub-xiphoid) approaches and percutaneous (transvascular and endocardial) approaches. Once access has been obtained, the composition may be delivered via epicardial, endocardial, or transvascular approaches. This may be done with appropriate catheters which one of ordinary skill would recognize need to be used for the above routes. The composition containing at least one PARP inhibitor may be delivered to the cardiac wall tissue in one or more locations. This includes intra-myocardial, sub-endocardial, and/or sub-epicardial administration.

One method to predictably deliver compositions containing at least one PAR inhibitor into such a moving target tissue is to time injections specifically for delivery during a select portion of the cardiac cycle. In one embodiment of the present invention, one or more electrodes may be used as stimulation electrodes, e.g., to pace the heart during delivery of composition. In this way, the cardiac cycle is made to be predictable and injection can be timed and synchronized to it. In fact, the beat-to-beat period can be artificially lengthened so as to permit complete delivery during a specific (and relatively) stationary phase of the cardiac cycle. In one embodiment, the delivery device includes one or more stimulation and/or sensing electrodes. In one embodiment of the present invention, sensors may be used to sense contractions of the heart, thereby allowing the delivery of composition to be timed with cardiac contractions. For example, it may be desirable to deliver one or more components of the PARP inhibitor composition between contractions of the heart.

Regardless of the method used to access a heart having a region of injured cardiac tissue or stabilize the heart, the delivery devices used may need to be capable of injecting multiple components separately into the cardiac wall. One embodiment of the current invention enables repeated injection by a single device. This may be achieved by a proximal one-hand trigger that enables predictable delivery of a determinable (e.g., dial-in) dose of a single- or multiple-constituent composition in a determinable ratio. A different embodiment of the current invention utilizes delivery devices having dual lumen needles/delivery catheters, and at least one other embodiment uses delivery devices having three or more lumen needles/delivery catheters. The lumens in the needles/delivery catheters can be in a coaxial configuration or a biaxial configuration.

Also, the cardiac tissue to which the at least one PARP inhibitor is delivered can be, for example, injured cardiac tissue, peri-injured cardiac tissue, and healthy cardiac tissue surrounding injured cardiac tissue. As used here, “delivery” refers to providing a composition to a treatment site in an injured tissue through any method appropriate to deliver the functional composition to the treatment site. Non-limiting examples of delivery methods include direct injection at the treatment site, direct topical application at the treatment site, percutaneous delivery for injection, percutaneous delivery for topical application, and other delivery methods well known to persons of ordinary skill in the art.

Injury area: As used herein, “injury area” refers to the injured tissue. The “peri-injury area” refers to the tissue immediately adjacent to the injured tissue. That is, the tissue at the junction between the injured tissue and the normal tissue. Injured tissue: As used herein, “injured tissue” refers to tissue injured by trauma, ischemic tissue, infarcted tissue or tissue damaged by any means which results in interruption of normal blood flow to the tissue. Related to the heart, “injured tissue” includes tissue undergoing any of the changes described under “cardiac tissue injury.”

The delivery system may deliver the components of the composition in a prescribed ratio. This ratio may be pre-set (and fixed) or dialable (and dynamic). One embodiment of the present invention utilizes separate gears or levers (with gear-ratio or lever-ratio that are settable) to enable delivery of multiple compounds in different ratios without generating a pressure gradient between syringes. Other multi component delivery devices of the current invention include lumens of different caliber to allow for pre-determined ratio of each component. Some multi-component delivery devices of the current invention include lumens of different lengths, such that one component is released more distally than another. Still other devices incorporate one or more mixing chambers in the device. At least one embodiment of delivery devices of the current invention includes single lumen needle/catheters that are used for serial delivery of multiple components (one after another)

A method of delivery of the at least one PARP inhibitor within the scope and teachings of the present disclosure is by epicardial, direct injection into cardiac tissue during an open chest procedure. Another approach, again within the scope and teachings of the present disclosure, is delivery of PARP into cardiac tissue via an intravascular approach. Catheters may be advanced through the vasculature and into the heart to inject materials into cardiac tissue from within the heart.

Several embodiments of delivery devices can be placed in a vessel neighboring the target treatment site and used to deliver PARP inhibitor compositions to the cardiac wall by piercing through the vessel wall and navigating to the desired location with the needle-tip or a microcatheter that is contained in the needle. The catheter or needle may contain a local imaging system for identifying the target area and proper positioning of the delivery device. The device may include one or more needles having a closed distal tip and one or more side openings for directing a substance substantially laterally from the distal tip into the cardiac wall. Preferably, the needle has a sufficiently small gauge diameter such that the needle track in the cardiac wall is substantially self-sealing to prevent escape of the composition upon removal of the needle. Recent data (obtained in the context of epicardial delivery) demonstrated hemostasis in vivo when PARP inhibitor gel was injected through even a large 18 gauge injection needle. This result could be attributable to the rapid coagulation achieved by the components injected and the inherent hemostatic properties of PARP inhibitor gel. In another embodiment, the needle gauge is smaller than 18 gauge. In one embodiment, the needle gauge is 26 gauge.

Alternatively, the delivery assembly may include one or more needles having a plurality of lumens that extend between a multiple line manifold on the proximal end to adjacent outlet ports. A multi-lumen needle assembly may allow components of a substance to be independently injected, thereby allowing the components to react with one another following delivery within the selected tissue region, as described herein.

In one embodiment, a multi-lumen needle assembly may allow two components of a composition to be simultaneously, independently injected, which may then react with one another once within the selected tissue region, as described herein. In another embodiment having a multi lumen needle assembly, the lumens empty into a mixing chamber located near the distal tip of the needle and the components of the injected substance are mixed with each other immediately prior to being injected into the selected tissue region.

In one embodiment of the current invention can be delivered to the cardiac wall by a catheter system. Catheter delivery systems suitable for the current invention include systems having multiple biaxial or coaxial lumens with staggered or flush tips. The catheter systems of the current invention can include needles or other injection devices located at the distal end, and syringes at the proximal end of the catheters. The catheters and other delivery devices of the current invention can have differently sized lumens to ensure that multi-component compositions can be delivered to the cardiac tissue in the desired ratio. Another embodiment of a catheter system may be used to create a composition reservoir within the cardiac wall itself to provide sustained delivery. A catheter may be introduced endovascularly into a blood vessel until the distal portion is adjacent the desired treatment location. The needle assembly may be oriented and deployed to puncture the wall of the vessel and enter the cardiac tissue. The composition can then be injected into the cardiac tissue and, thereby, form a reservoir. When catheter systems are used, a clinician can navigate to a patient's heart using one of the plurality of routes known for accessing the heart through the vasculature, or navigation to a heart chamber for delivery of the compositions epicardially, endocardially or transvascularly.

A clinician practicing the current invention may need to make multiple injections using a single delivery assembly. Thus, at least one embodiment of the delivery devices of the current invention includes a device having at least one reusable needle. Some embodiments of the present invention may include delivery devices having an automated dosing system, e.g., a syringe advancing system. The automated dosing system may allow each dose to be pre-determined and dialed in (can be variable or fixed), e.g., a screw-type setting system. One embodiment of the current invention may include a proximal handle wherein each time the proximal handle is pushed; a pre-determined dose is delivered at a pre-determined or manually-controllable rate.

In further alternative embodiments, the delivery system may include a plurality of needle assemblies (similar to the individual needle assemblies described above), to be deployed in a predetermined arrangement along the periphery of a catheter. In one embodiment, the needle assemblies may be arranged in one or more rows. In particular, it may desirable to access an extended remote tissue region, for example extending substantially parallel to a vessel, within the myocardium. With a multiple needle transvascular catheter system, a single device may be delivered into a vessel and oriented. The array of needles may be sequentially or simultaneously deployed to inject a composition into the extended tissue region, thereby providing a selected trajectory pattern. Catheter based devices such as those described above are disclosed in U.S. Pat. No. 6,283,951, the disclosure of which is incorporated herein by reference thereto.

If a clinician is practicing the current invention using a minimally invasive or percutaneous technique, he/she may need some sort of real-time visualization or navigation to ensure site-specific injections. Thus, at least one embodiment of the present invention uses MNav technologies to superimpose pre-operative MRI or CT images onto fluoroscopic images of a delivery catheter to track it in real-time to target sites. In one embodiment, the clinician uses a contrast agent and/or navigation technologies to track the needle-tip during injection in a virtual 3-D environment. This technique marks previous injections to ensure proper spacing of future injections.

The needle assembly (or other device component) may include a feedback element or sensor for measuring a physiological condition to guide delivery of compositions to the desired location. For example, an EKG lead may be included on the distal tip or otherwise delivered within the selected tissue region to detect and guide injection towards electrically silent or quiet areas of cardiac tissue, or to allow electrical events within the heart to be monitored during delivery of the composition. During treatment, for example, the composition may be delivered into a tissue region until a desired condition is met. Also, local EKG monitoring can be used to target and guide injection towards electrically silent or quiet areas of cardiac tissue.

Regardless of the device used to deliver the PARP inhibitor composition or how the clinician accesses the cardiac wall, a clinician practicing the current invention may have the need for precise local placement and depth-control for each injection. In one embodiment of the present invention, the substance is delivered/injected to a depth in the cardiac wall that is approximately midway between the outside wall and the inside wall. In other embodiments, the substances are delivered to a depth that is closer to either the inside wall or the outside wall. The substances may be delivered intra-myocardially, sub-endocardially, or sub-epicardially. In another embodiment of the invention, the depth of the injection will vary based on the thickness of the target tissue and the depth is less at the apex of a heart than it is at other locations on the heart.

To achieve depth control, the delivery device of at least one embodiment of the present invention includes a stopper fixed (or adjustably fixed) on the needle shaft, at a desired distance from needle's distal tip, to prevent penetration into tissue beyond a specified depth. Some embodiments use the method of injecting one or more needles into tissue at a tangent to the tissue surface to control the depth of the injection. In at least one embodiment of the present invention, the needle can be positioned to inject at an angle perpendicular (90 degrees) to the tissue, tangential (0 degrees) to the tissue, or any desired angle in between. Suction can facilitate controlled positioning and entry of the injector.

At least one embodiment of the present invention uses a “Smart-Needle” to detect distance from the needle tip to the ventricular blood compartment or endocardial surface, so that the needle tip is maintained in the cardiac wall. Such a needle can rely on imaging around or ahead of the needle tip by imaging modes such as ultrasound.

At times it might be desirable to distribute the PARP inhibitor composition as widely as possible around the injection site. It might also be desirable to have the PARP inhibitor composition be uniformly distributed around the injection site. One method for enhancing distribution of a PARP inhibitor composition around an injection site is to use needles having holes in the side vs. using needles having holes in the end. Multiple side holes can provide a wider distribution of composition around the injection site. Side holes also provide access to the tissue from a multitude of places rather than just from the end of the needle, thereby requiring less travel of the composition for wider distribution. A potential benefit of side holes in the needles is that if the needle tip accidentally penetrates through the heart wall and into a cardiac chamber, the composition may still be injected into cardiac tissue as opposed to being injected into the blood stream within the cardiac chamber. Another method for enhancing distribution of a composition around an injection site is to increase the number of needles used at the injection site. If desired, the multi-needle delivery device of the present invention, allows for multiple needles to be placed close to each other in order to provide a uniform distribution over a larger area as compared to the use of a single needle device. The combination of side holes on the needles of a multi-needle device may provide a broad distribution of composition around an injection site.

In one embodiment of the present invention, suction may be used to improve the distribution of a composition around the injection site. The use of suction can create a negative pressure in the interstitial space. This negative pressure within the interstitial space can help the composition to travel farther and more freely, since the composition is driven by a negative pressure gradient. The combination of suction and side holes on the needles of a multi-needle device may provide a more thorough and broad distribution of composition around an injection site.

In one embodiment of the present invention, the delivery of compositions containing at least one PARP inhibitor from the delivery device into tissue may be enhanced via the application of an electric current, for example via iontophoresis. In general, the delivery of ionized agents into tissue may be enhanced via a small current applied across two electrodes. Positive ions may be introduced into the tissue from the positive pole, or negative ions from the negative pole. The use of iontophoresis may markedly facilitate the transport of certain ionized agents through tissue.

In one embodiment, one or more needles of the delivery device may act as the positive and/or negative poles. For example, a grounding electrode may be used in combination with a needle electrode via a monopolar arrangement to deliver an ionized composition iontophoretically to the target tissue. In one embodiment, a composition may be first dispersed from the needle into tissue. Following delivery, the composition may be iontophoretically driven deeper into the tissue via the application of an electric current. In one embodiment, a delivery device having multiple needles may comprise both the positive and negative poles via a bipolar arrangement. Further, in one embodiment, multiple needle electrodes may be used simultaneously or sequentially to inject a substance and/or deliver an electric current.

When practicing the current invention, one goal is to inject a substance into the cardiac wall while avoiding accidental delivery into one or more chambers of the heart, the coronary artery or venous system. Delivery into one or more of these areas may have negative consequences such as pulmonary or systemic embolization, stroke, cardiac congestion, and/or distant thromboembolism, for example. The current invention addresses and attempts to prevent these negative consequences in a variety of ways. In at least one embodiment of the present invention, the ratio of the components of the composition is selected so that the composition gels or polymerizes almost immediately in-situ to minimize migration of one or more of the components. In one embodiment, a balloon catheter is placed in the coronary sinus and inflated during delivery until gelling is complete. This would prevent liquid components from traveling from the tissue to the coronary venous tree and instead promote residence and gelling in the target tissue. At least one embodiment includes a pressure control system on the delivery device, to ensure that injectate pressure never exceeds ventricular chamber pressure. This would encourage retention in tissue and prevent pressure-driven migration of the composition through the thebesian venous system into the cardiac chamber. One embodiment of the present invention uses a “Smart Needle” as described above to prevent negative consequences from occurring.

At least one embodiment of the present invention includes a proximally-hand-operated distal sleeve that covers the needle tip or applies local negative pressure to prevent outward flow of component(s) from the tip of the needle between injections where multiple injections are required. In at least one embodiment, the column of components in a catheter is held under a constant minimum pressure that prevents outflow in between injections. In at least one embodiment, one-way valves may be placed within each line to prevent entry of one component into a line containing another. This is especially important when the gelling reaction is rapid and the different components need to be maintained separately until the time and site of injection. This will prevent clogging of the delivery device, which will allow repeated injections using a single device.

At least one embodiment of the present invention prevents backbleed out of the needle track, during and after removal of the needle, by keeping the needle in place for several seconds (e.g. 5-30 sec beyond the expected clotting time) following injection, to utilize the injectate as a ‘plug’ preventing back-bleed, before removing needle. In at least one embodiment of the current invention, the needle is left in place for the expected gelling time of the injected substance and then withdrawn. In one embodiment of the invention, the gelling time of an injected composition is five seconds.

Several embodiments of the current invention can include sensors and other means to assist in directing the delivery device to a desired location, ensuring that the injections occur at a desired depth, ensuring the delivery device is at the treatment site, ensuring that the desired volume of composition is delivered, and other functions that may require some type of sensor or imaging means to be used. For example, real-time recording of electrical activity (e.g., EKG), pH, oxygenation, metabolites such as lactic acid, CO₂, or other local indicators of cardiac tissue viability or activity can be used to help guide the injections to the desired location. In some embodiments of the present invention, the delivery device may include one or more sensors. For example, the sensors may be one or more electrical sensors, fiber optic sensors, chemical sensors, imaging sensors, structural sensors and/or proximity sensors that measure conductance. In one embodiment, the sensors may be tissue depth sensors for determining the depth of tissue adjacent the delivery device. In one embodiment, a sensor that detects pH, oxygenation, a blood metabolite, a tissue metabolite, etc may be used at the end of the delivery device to alert the user if and when the tip has entered the chamber blood. This would cause the operator to re-position the delivery instrument before delivering the composition. The one or more depth sensors may be used to control the depth of needle penetration into the tissue. In this way, the needle penetration depth can be controlled, for example, according to the thickness of tissue, e.g., tissue of a heart chamber wall. In some embodiments, sensors may be positioned or located on one or more needles of the delivery device. In some embodiments, sensors may be positioned or located on one or more tissue-contacting surfaces of the delivery device. In other embodiments of the present invention, the delivery device may include one or more indicators. For example, a variety of indicators, e.g., visual or audible, may be used to indicate to the physician that the desired tissue depth has been achieved.

Furthermore, the delivery device may comprise sensors to allow the surgeon or clinician to ensure the delivery device is within the heart wall rather than in the ventricle at the time of injection. Non-limiting examples of sensors which would allow determination of the location of the injector include, pressure sensors, pH sensors and sensors for dissolved gases, such as oxygen. An additional sensor that may be associated with the delivery devices suitable for use with the present invention include sensors which indicate flow of blood such as a backflow port or a backflow lumen which would inform a surgeon or clinician that the needle portion of the delivery device is in an area which has blood flow rather than within a tissue.

The total injection volume per heart may be dose-dependent based on different factors such as the size of the heart and the size of the injured region of cardiac wall.

The number of injection sites per heart can be based on the size and shape of the injured region, the desired location of the injections, and the distance separating the injection sites. In at least one embodiment, the number of injection sites can range from 5-25 sites. The distance separating injection sites will vary based on the desired volume of PARP inhibitor composition to be injected per injection site, the desired total volume to be injected, and the condition of the injured tissue. In at least one embodiment, the distance between injection sites is approximately 2 cm and in at least one other embodiment, the distance between injection sites is 1 cm. In still another embodiment, the separation distance between injection sites can range between about 50 mm and about 2 cm. In another embodiment, the distance between injection sites can be in the range of 0.5 cm to 2.5 cm. In another embodiment, the distance between injection sites is greater than 2.5 cm. Injections can be continuous or interrupted along a needle track instead of as discrete single injections.

In one embodiment of the present invention, the composition containing at least one PARP inhibitor is injected into the cardiac tissue in a pattern that encourages formation of blood vessels. One exemplary pattern is a linear pattern that connects two target areas of tissue so that formation of blood vessels is stimulated along the linear pattern. In another embodiment, the pattern is branched. In particular, the formation of blood vessels comprises the formation of large-bore conduit vessels

In another embodiment, more than one composition can be injected into a treatment site.

The location of the delivery can vary based on the size and shape of the injured region of cardiac tissue, and the desired extent of structural reinforcement of the tissue. In at least one embodiment of the present invention, the composition is delivered only into the injured cardiac tissue, while in other embodiments the peri-injury zone around the injured region is treated, and, in at least one other embodiment, the composition is delivered into only the healthy tissue that borders an injured region. In other embodiments, the composition may be delivered to any combination of the regions of injured cardiac tissue, tissue in the peri-injury zone, and healthy tissue.

The timing of PARP inhibitor composition delivery relative to an injurious event will be based on the severity of the injury, the extent of the injury, the condition of the patient, and the progression of any tissue remodeling. In at least one embodiment, the PARP inhibitor composition is delivered one to eight hours following an injurious event such as an MI, for example within one to eight hours following ischemia-reperfusion (in the catheterization lab setting immediately after re-perfusion). In another embodiment, the PARP inhibitor composition is delivered to the cardiac wall within one hour of an injurious event. In another embodiment the PARP inhibitor composition is injected three to four days after an injury (after clinical stabilization of the patient, which would make it safe for the patient to undergo a separate procedure). In at least one embodiment, the PARP inhibitor composition is delivered more than one week after the injury, including up to months or years after injury. Other times for injecting compositions into the cardiac wall are also contemplated, including prior to any injurious event, and immediately upon finding an area of injured cardiac tissue (for preventing additional remodeling in older injuries). In another embodiment of the invention, compositions can be injected into the cardiac tissue years after an injurious event. In another embodiment, the PARP inhibitor composition is injected into the cardiac tissue from about 1 hour to about 2 years after an injurious event. In another embodiment, the PARP inhibitor composition is injected into the cardiac tissue from about 6 hours to about 1 year after an injurious event. In another embodiment, the PARP inhibitor composition is injected into the cardiac tissue from about 12 hours to about 9 months after an injurious event. In another embodiment, the PARP inhibitor composition is injected into the cardiac tissue from about 24 hours to about 6 months after an injurious event. In another embodiment, the PARP inhibitor composition is injected into the cardiac tissue from about 48 hours to about 3 months after an injurious event. In another embodiment, the PARP inhibitor composition is injected into the cardiac tissue up to about 10 years after an injurious event.

The present PARP inhibitors may be delivered to locations within the human vascular system with the use a catheter in addition to cardiac muscle. The catheter can be a balloon catheter which in one embodiment is a dual balloon catheter. The PARP inhibitors may be delivered during deployment. They may also be delivered by using a stent or stent graft. These can be used alone or currently in conjunction with a catheter delivery system.

In addition to the foregoing uses for the PARP inhibitor compositions, methods and systems of the present invention, it will be apparent to those skilled in the art that other injured tissues, in addition to injured cardiac tissue, would benefit from the delivery of a treatment that promotes neovascularization. Examples of such tissues include ischemic tissues in organs or sites including, but not limited to, wounds, gastrointestinal tissue, kidney, liver, skin, and neural tissue such as brain, spinal cord and nerves.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A system for treating cardiac tissue comprising:
a composition comprising at least one poly(ADP-ribose) polymerase (PARP) inhibitor;
and at least one delivery device for introducing said composition into said cardiac tissue.

2. The system of claim 1, wherein said at least one PARP inhibitor is a PARP-1 inhibitor or a PARP-2 inhibitor.

3. The system of claim 1, wherein said at least one PARP inhibitor is INO-1001 or BGP-15.

4. The system of claim 1, wherein said cardiac tissue is previously injured.

5. The system of claim 1, wherein said composition is introduced into said cardiac tissue during an injurious event or after an injurious event has occurred.

6. The system of claim 5, wherein said event is an ischemic event.

7. The system of claim 1, wherein said composition is introduced to said cardiac tissue at the location of and during revascularization.

8. The system of claim 1, wherein said cardiac tissue is selected from the group consisting of injured cardiac tissue, peri-injured cardiac tissue, and healthy cardiac tissue surrounding injured cardiac tissue.

9. The system of claim 1, wherein said delivery device is an injection catheter selected from the group consisting of an endocardial injection catheter, a transvacular injection catheter and an epicardial injection catheter.

10. The system of claim 1, wherein said delivery device is a stent or stent graft.

11. The system of claim 1, wherein said composition further comprises a bioactive agent.

12. The system of claim 4, wherein introduction of said composition into said injured cadiac tissue is via an injection site on said injured cardiac tissue.

13. The system of claim 12, wherein said injection site in said injured cardiac tissue is selected from the group consisting of sub-endocardial, sub-epicardial and intra-myocardial sites.

14. The system of claim 11, wherein said bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof.

15. The system of claim 1, wherein said composition further comprises a contrast agent.

16. The system of claim 1, wherein said composition is provided to said injured cardiac tissue between about 1 hour and about 1 year after injury occurs to said cardiac tissue.

17. The system of claim 1, wherein said composition is provided in about 1 to 20 injections.

18. The system of claim 17, wherein said injections are provided sequentially.

19. The system of claim 17, wherein said injections are provided approximately simultaneously.

20. A method for treating cardiac tissue comprising:
delivering a composition comprising at least one poly(ADP-ribose) polymerase (PARP) inhibitor to said cardiac tissue of a patient in need thereof with at least one delivery device for introducing said composition into said cardiac tissue.

21. The method of claim 20, wherein said at least one PARP inhibitor is PARP-1 inhibitor or PARP-2 inhibitor.

22. The method of claim 20, wherein said at least one PARP inhibitor is INO-1001 or BGP-15.

23. The method of claim 20, wherein said cardiac tissue is previously injured.

24. The method of claim 20, wherein said composition is introduced into said cardiac tissue at the location of and during revascularization.

25. The method of claim 20, wherein said composition is introduced into said cardiac tissue during an injurious event or after an injurious event has occurred.

26. The method of claim 20, wherein said cardiac tissue is selected from the group consisting of injured cardiac tissue, peri-injured cardiac tissue, and healthy cardiac tissue surrounding injured cardiac tissue.

27. The method of claim 20, wherein said delivery device is an injection catheter selected from the group consisting of an endocardial injection catheter, a transvascular injection catheter and an epicardial injection catheter.

28. The method of claim 20, wherein said delivery device is a stent or stent graft.

29. The method of claim 20, wherein said composition further comprises a bioactive agent.

30. The method of claim 20, wherein introduction of said composition into said injured cardiac tissue is via an injection site on said injured cardiac tissue.

31. The method of claim 30, wherein said injection site in said injured cardiac tissue is selected from the group consisting of sub-endocardial, sub-epicardial and intra-myocardial sites.

32. The method of claim 29, wherein said bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof.

33. The method of claim 20, wherein said composition further comprises a contrast agent.

34. The method of claim 20, wherein said composition is provided to said injured cardiac tissue between about 1 hour and about 1 year after injury occurs to said cardiac tissue.

35. The method of claim 20, wherein said composition is provided in about 1 to 20 injections.

36. The method of claim 35, wherein said injections are provided sequentially.

37. The method of claim 35, wherein said injections are provided approximately simultaneously.
